(19) United States
(12) Patent Application Publication
(10) Pub. No.: US 2011/0004114 A1
(43) Pub. Date: Jan. 6, 2011

(54) Cognitive information encoding conversion telecommunication system and conferencing method

(75) Inventor: Farida Hanna Schenck, New York, NY (US)

Correspondence Address: Mrs. Farida Hanna Schenck, P.O. Box 3513, New York, NY, 10185, US

(73) Assignee: Farida Hanna Schenck (Campbell),, New York, NY (US)

(21) Appl. No.: 12/459,636

(22) Filed: Jul. 6, 2009

(51) Int. Cl. A61B 5/0476

(52) U.S. Cl. 600/544

(57) ABSTRACT

This invention relates to a scanless system for- and method of teleportation of cognitive perceptions as events in series in conjunction with- or without radio-telephone frequency applications. In the invention, an event includes the focal content of bound percepts in a source-subject. The invention describes the process whereby the quantum nature of the information-bearing content of those percepts, whether founded on physical or mental (imaginary) stimuli can be transceived through the invention as concept modules whose origins lie within the data-free elements of sensory organ electromagnetic radiation frequency wave responses to the source-subject's individually-directed and subjectively-generated emotions. At the core, the assembly identifies a method for capturing the engagement between photon-carrying information and transmissions to the receiving-subject or -devices using spintronic-based evaluation for a plurality of inputs and a plurality of outputs with a radio-frequency telephone-enabled assembly, governed by quantum key distribution protocols and time-dependent delay processors.

### INVENTION DESCRIPTION

1. Field of the Invention

This invention relates to a METHOD of quantum cognitive conversion, which incorporates quantum cryptographic and cognitive information encoding conversion via modular-based conferencing of information that is produced and controlled by the source-subject's emotions in order to produce cognitive perceptions (from learning, memory, physical or mental triggers) that are mirrored in the receiving priority sensory organs (eyes, ears, etc.).

2. Discussion of Background

The background of the invention is explained in three fundamental sections: the Utility of the invention in terms of current applications involving Neuronal sensory electromagnetic radiation and Cognitive event processing; models for capturing sensory perception information-content and delineation of events according to subjectively-driven modular concepts; and, the application of quantum cryptography, quantum computation in order to achieve a scanless virtual device capable of receiving and transmitting fundamentally irregular but quantum-accurate optical and auditory information.

### Utility of the Invention: Neuronal Sensory Electromagnetic Radiation and Cognitive Event Processing

Attention to any given cognitive perception is currently based on non-invasive experimental electrode evaluation of pronounced action potentials, known as “spikes” from different response regions of the brain.

The receptive field of a sensory neuron can vary in time and according to a sensory specific dimension, such as space for vision and frequency of sound for auditory.

The term Spatio-Temporal Receptive Field (STRF) can be used to describe these fields and the mathematical descriptions of active norms in natural daily stimuli tend to be complex.

The organization of these spikes in terms of so-called Event-related potentials (ERPs) provides valuable clues regarding the brain dynamics sub-serving cognitive functions such as attention and working memory (Barcelo, Perianez, Knight).

Emotions essentially drive the process of perception-building in cognitive functioning. According to Klimesch and Wolfgang, it is established that brainwave oscillations play a causal role for cognitive performance including phase locking and the timing of cognitive processes.

Emotions substantiate their execution and the logic derived for the information values contained within conscious cognitive performance is a subjective unpredictable process of personal intention (“free will”), e.g., for a particular focus and in the transitions from one area of focus to another in memory or learning.

Perception visualization or hearing however actually occurs at the primary sensory organ, which is engaged with the various brain centres involved in stimulating these organs to effectively ‘mirror’ information at will and in advance of secondary responses to the information (information series) such as verbal or physical movement.

Physically, basilar membrane shakings in the ear because of sound waveforms entering into the ear setup nerve action to related of hearing—even of one's own thoughts such as during reading and writing.

The eye retina is part of the optic nerve that transmits optic impulses to the brain. Light passes through the lens, pupil and cornea on its way to retina where the visual scene is focused). The lens projects an inverted image onto the retina. Information flows vertically from photoreceptors of the retina to bipolar cells to ganglion cells, as well as laterally via horizontal cells in the outer plexiform layer and amacrine cells in the inner plexiform layer. The output of the retina is converted by the ganglion (neuron) cells which“ . . . transmit information as trains of action potentials” (Kandel, Schwartz, Jessel, 1991. p 524.). The bipolar, horizontal and amacrine cells transmit signals from the photoreceptors to the ganglion cells and combine signals from several photoreceptors in such a way that the electrical responses evoked in the ganglion cells depend critically on the precise spatial and temporal patterns of the light that stimulates the retina. There is a difference between visual image and retinal image. The brain is an active information processing organ that is capable of regenerating “mirror” replicas with subjective degrees of accuracy, depending on the subject's experience, age, and other factors. The invention capitalizes on the fact that the brain effectively uses the sensory organ as an opportunity to mirror a “re-experience” of a perception or deductions about a previous perception, for a fleeting instant of recollection. This means that the mirroring process that takes place will induce response behaviours at the location where the image appears in the visual field at the retinal ganglia. For clinical and experimental purposes, the surface of the retina is divided into sections relative to the midline of the normal standard field of vision which can be used for reference comparison to the invention output deliverables. It is established that each optic tract carries a complete representation of one half of the binocular zone in the visual field. For this reason, the invention requires both eyes to be included ideally.

Light rays from any object radiate from any object, the angle into our eyes grows smaller and smaller. Beyond 15 feet the rays are practically parallel. Parallel rays produce sharp image without any eye-effort (complete relaxation). As an object approaches, the rays' angles increase and the eye automatically adjusts itself physiologically to accommodate clear image perception. Light falling on retina is coded and relayed to visual cortex. In the invention, the electromagnetic responses to produce perceptions triggered and organized by the brain at the location of the optical sensory organs is used to track those cognitive perceptions and transmit them to receiving devices or receiver-sources. Just as light falling on the retina stimulates the fibres on the optic nerve, the method of the assembly is stimulated in the presence of acute differentiable emissions from the retinal neurons. Because the primary visual cortex is responsible for the position of objects in space as well as their relationship to each other, as well as the perception of light and shade, the maximum information is derived from both eyes (in humans) or all optical-priority organs. (in other living animals). In this way an overall composite image of any object is formed. While interpretation of image occurs in the brain, interpretation in the invention takes place in the assembly of the receiver-source instantaneously because of the nature of information teleportation directly to their brain and/or the incorporation of methods for the computational analysis, resources and output video or acoustical handling in established cryptographic networks (see U.S. Pat. No. 7,460,670 B1).

### Modular-Concept Embodiment of Detailed Sensory Perception Information-Content

Techniques to decode the visual and subjective contents of the human brain have tended to focus on statistical algorithms to assess fMRI-based signal activity and to extrapolate and/or classify various brain states for identified stimulus. They do not presently scope the specific non-stationary independent and unpredictable information content of cognitive perceptions attributed to conscious brain activity and which to-date, remain invisible or unknown to the experimental assessor of a given source-subject (Kamitani, Tong).

Other studies and practices are designed to extrapolate and predict “intentions” based on the differences in neuron orientation, excitation and comparative ERP in normal functioning versus disorder or disease. However, these still do not claim to recognize or predict what a person is perceiving in the random, day-to-day world let alone present a mechanism by which to communicate these perceptions in real-time to remote devices or third-party receiver-sources.

Psychological and psychosensory evaluations collectively depend on data that is external or verbal such as physiological tests and scans combined with questions and survey responses from the source-subject. These include eye-movement, body temperature fluctuations, etc.

In the invention, various versions of event-definition and perception-information extrapolation is based on a variety of media (thought-formation). This can include enhanced visual processing or auditory components.

The term “qubit” refers to individual two-state quantum subsystem which carries the spinor information of an electromagnetic brain wave and which is involved in mirroring for teleportation. Decoherence represents losses to the qubit to the environment and the use of specific quantum codes found in other patented technologies including U.S. Pat. No. 6,128,764 to Gottesman and U.S. Pat. No. 7,184, 555 to Magiq Technologies serves to explain solutions.

According to the Pauli Exclusion Principle, the information encoded in brainwaves cannot be identical on separate waves simultaneously in the same location and therefore perceptions are never the same or simultaneously “doubled” for an individual subject. Modular concepts add or subtract in a variety of fashions. At the electromagnetic level, spin is a quantized property and entanglement occurs when two or more particles come into contact and their wave properties become identical and remain as such as long as they are not disturbed—regardless of the distance between them. Disturbance, on the other hand, leads to wave function collapse at both locations simultaneously. This represents the principle by which the brain and bound percept transformations between changing brainwaves. The assembly of the invention centres its activation and conversions of coded information from these mirrors, optically from the eyes and acoustically from the ears. The data from the eyes and ears combines to form a multi-dimensional ‘bound percept’ derived from oscillations that resonate as a mixed transformation of topological surfaces (manifolds) bearing spinor information that is teleported between neurons and the sensory organs simultaneously and according to entanglement principles of quantum information borne on electromagnetic waves of the brain. By the standards of this invention, sense data that is naturally therefore available as the substrate of the percept both to the source-subject brain becomes additionally available to one or more remote receiver-subjects and/or storage and programming devices regardless of those receiver-subjects locations in time or space.

Peak sensitivity for specific feature of a stimulus (Boynton, 2005. P. 465.) within a neuron can be depicted by:

$$H ( {x}_{x} , {c}_{i} ) = \frac{{\sum }_{i} {( {c}_{i} F ( {x}_{i} ) )}^{2}}{{\sum }_{i} {c}_{i}^{2} + {σ}^{2}}$$

According to Boynton:

“The total excitatory response in the numerator is the sum of squared linear responses to each stimulus component. The inhibitory response in the denominator is the sum of squared contrasts plus a semisaturation term, (s). This term in the denominator corresponds to the contrast energy of the stimulus and represents a divisive input from a population of neurons selective across the spectrum of feature space [7]. This model predicts an S-shaped, or sigmoidal, contrast-response function that is suppressed divisively when non-preferred stimuli are added to the RF. The model correctly predicts that increasing stimulus contrast increases neuronal responses without altering the selectivity of a neuron for a given feature, such as orientation or spatial frequency . . . ”

This firing sequence is what defines the orbital transgression of emitting light or energy particulates (photons) which carry information. With this in mind, arrays of information portray the transformation of existing perception to emerging perception, and homologically defined by constant curvatures and topology characterizations such as those using Riemannian manifolds or Penrose' Twistor space model.

Studies involving rapidly changing scenes show that the percept derives from numerous processes that each involve time delays (see Moutoussis and Zeki (1997)). Considering that the visual information is encoded on the electromagnetic waves generated by the source-subject brain, conversion of the information encoding takes place as an awareness of a perception which is mirrored optically at the location of electrical-activity by and among neurons at the receiving end of the retina. It is rendered by the invention's capacity to detect those pieces of information (spinors) from the waves that carry them there which it does so by merging sensitivity to the output of these waves with an alignment to the characteristic properties and functionalities of the wave components using the phenomenon of both wave exploration and wave management in the features of the design. As this begins to take place, a disruption in the service execution falls down and delivers—by momentum or gravity which has the same effect as producing or restoring a new alignment so that the process can begin anew or enhanced with the integration of the information transmission sent just previous.

In the case of using Riemannian geometry, for example, curvature is explained by an unknown author (http://www.mathpages.com/rr/rrtoc.htm):

An N-dimensional Riemannian manifold is characterized by a second-order metric tensor gₘₙ(x) which defines the differential metrical distance along any smooth curve in terms of the differential coordinate components according to

(ds)²=g_{μν}(x)dx^{μ}dx^{ν}

where, as usual, summation is implied over repeated indices in any product the metric components as gₘₙ(x) to emphasize that they are not constant, but are allowed to be continuous differentiable functions of position.

The method of this invention is centrally based on responding to the complexifications that take place in producing perceptions at the site of the sensory faculties. In the derivation of the method, the inventor/author made frequent initial use of Sir Roger Penrose' quantum twistor mathematical theory and harmonic maps from Riemann surfaces to describe the output projections from the neurons' electromagnetic wave phenomenon and how they were able to conference meaningful cognitive perceptions.

In his own words (Penrose, Hadrovich.

Twistors are essentially complex objects, like wavefunctions in quantum mechanics, as well as endowed with holomorphic and algebraic structure sufficient to encode space-time points. In this sense twistor space can be considered more primitive than the space-time itself and indeed provides a background against which space-time could be meaningfully quantised. In quantum mechanics a state of an object is described by a vector in Hilbert space. This is a linear superposition of some basis vectors, for example, in the case of an electron (or any spin—½ particle) it is some linear superposition of the spin “up” and “down” states. The two component vector (z,w) is called a spinor. The norm of this spinor caries no information-indeed, it is usually set to 1. Only the direction is physically significant which forces us to consider the projective (spin) space. In the electron example, spin state is therefore described by a point in CP1, conveniently represented with Riemann sphere. Stereographic projection is a shape (i.e. angle) preserving map. Hence circles on the sphere will be mapped to circles on the plane (except those containing the south pole, which are mapped to straight lines on the plane). This geometry is preserved by Möbius (fractional linear) transformation. In terms of spinors, this is the action of SL(2,C). In relativity, an observer sees a t=const. section of its past null cone. This is a celestial (Riemann) 2-sphere that can be stereographically projected onto a complex plane. Möbius transformations of the plane correspond to Lorentz transformations of the celestial sphere. This “spinorial” point of view has an interesting application in finding the apparent shape of a rapidly moving sphere.

Considering “thought” therefore, the functional relationship between contour geometric wave evaluation and information sensitivity is handled perfectly by a system that makes use of the above or similar mappings of information encoded on the waves.

### Quantum Cryptography, Method of Computation and Scanless Virtual Optical and Auditory Information Conversion Principles

At the core of the invention lies the response to automatic sensory-perception information codifications on electromagnetic waves discharged by the brain to localized sensory organs. Sensitivity to this information begins with spin-wave architectures that have been modified to collect as well as provide the physical mechanism of the device inter-connection. In addition, at the site of each sensory organ a non-invasive receiver receives information through scanless conversion multi-component time-dependent modules; a decoder for decoding received electrical signals, and an optional modulator for driving the perception event-processing such as to a radio-frequency telephone device. In the invention, this may include an assembly system containing a hyperfine pulsating radio-frequency modulation system radiating within the electrical signal-processing surrounding the detectors. Such hyperfine states of trapped ions found therein are used for storing qubits in ion-trap quantum computings which last up to 10 minutes. The frequency associated with the states' energy separation is derived from pulses produced by elevating radio-telephone or other radiation at least to microwave or infrared levels. Modulated radio-telephone (or other) waves pulse in pairs through the system in order to achieve a stable system for mirroring and quantum computation of the information on source waves from the brain or sensory organs attached to the brain.

These and other objects of the invention comprise the steps of defining a space (Hilbert, Minkowski or other) to represent a physical quantum mechanical system, selecting a set of quantum observables for said system, selecting a subspace of said space in which all states representing said quantum mechanical system have the same eigenvalue; and storing information in or processing information using said subspace.

Electromagnetic waves can be used to drive the evaluation process for each of the waves' positions. This is done through modulation of radio-telephone devices to bring or push the radiofrequency to low microwave radiation frequency regions. In addition, the process uses a combination of simultaneous effects generated from but not limited to:
neodymium magnet (also known as NdFeB magnet or Neo magnet) or similar element in filament or powder form, with exceptionally high uniaxial magnetocrystalline anisotropy and the potential for storing large amounts of magnetic energy (BHmax˜512 kJ/m3 or 64 MGOe.) in order to accommodate the conferencing time-dependencies between the modular-segments of the process.
an improved decoherence free subsystem defining a space (Hilbert, Minkowski or other) to represent a physical quantum mechanical system divided into subspaces according to the eigenvalues of defined wave-related observables (referred to in the diagrammes as the “conferencing disc”). This space may be filled with Helium or similar characteristic medium. The isotope helium-4 exists in a normal colorless liquid state, called helium I. According to Fred M. Ellis—Director, Department of Physics, Wesleyan University, Middletown, Conn. 06459 “In the low temperature regime, below 2.17 K at SVP, helium liquid is dominated by a “zero point” quantum energy which completely changes the behavior of the fluid. It is actually best thought of as a Bose-condensed many-particle system with every atom in the fluid occupying a single quantum state. With all of the particles sharing same state, and contributing collectively to the state's quantum energy level, single particle atomic scale interactions become impossible, essentially overwhelmed by the macroscopic quantum state changes that are favored by the huge numbers of particles involved. “(Source: https://wesfiles.wesleyan.edu/home/fellis/web/research/q_f_lab.html Jun. 15, 2009.)
a processing membrane feature whereby information encoding is identified and envelopes the wave characteristics as a plurality of input signal-receivers collected along and throughout the each wave and sequenced in a time-dependent network management in a cryptographic-type conferencing network that monitors parameters associated with multiple links and multiple nodes based on the monitored parameters (such as the one described by U.S. Pat. No. 7,460,670 BBN Technologies Corp., MA).
The effect of each transformation for a given wave component is different from any previous transformation on subsequent components.

### Sample Output

1. virtual sensory mirroring representations for sensorial structures, iconic memory representations versus iconic memory units, feature representations versus feature units, formal representations versus formal modules, conceptual representations versus conceptual modules, conceptual memory representations versus conceptual memory modules, propositional representations versus predication structures each of which are derived from:

2. Optical and auditory information tracking based on formulae for the lorentz attractor and its connection to the modular flow in order to follow the topological arrays and sequence of information events involved in concept transformations that take place in active thinking or learning. In the invention, each cognitive neural module will have a unique, natural, characteristic activation pattern that is derived from both the arrangement and the characteristic spiking frequencies of the individual neurons which altogether feed elementary concept module in realtime perceptions. Therefore, the module will be defined by a corresponding unique characteristic activation wave pattern and that propagated wave patterns constitute collectively the active thoughts of a source-subject. Such a wave pattern follows standard physical laws of resonance and superposition with regards to adjacent and emerging concept-based wave behaviour. At the region of the activated sensory organ these become quickly translated into efficient processing powers for perception-response and linguistic-thinking or other responses by the source-subject. In turn, the invention delivers specific and non-specific sensory perception from the source-subject. Modularity of concepts is entirely subjective and non-uniform. Minimal processing of sensory modules of information is never-ceasing in the living brain, i.e., it occurs even during sleep. (vander Nat, p. 10.) Temporary links pervade the relationship between existing and replacement of concept modules for the information transfer. However, this permits concept information can be broadcast to processing units within the receiver-source.

3. Routines are based on the means of extracting information from a visual and/or auditory cognition process. This may be bottom-up or top-down, during which each stage of the representations generated in the receiver-source correspond to retinotopic maps for properties like colour, motion, word, etc. or for auditory operations. Information-extraction routines are based on non-uniformly distributed visual cognition elements and only applied to objects or areas specified by the source-subject. In the subject-source, this represents shifting the processing focus, indexing a salient item for further processing, spreading activation over an area delimited by boundaries, tracing boundaries and marking a location or object for future reference. When combined into the tourines of the modular-components, these elementary operators can be used to perform relatively sophisticated spatial tasks by the source-receiver or receiving computer or AI programs (such as counting the number of objects or recognizing specific properties).

4. Peripheral, motor or remote technology activation resources originating from or along with the plurality of outputs from the assembly or via receiver-subject self-elected control of the assembly and confined to the teleportation of perception-driven retinal images (retinotopy) and/or resonating with similar derivations for the topological transforms that describe successful teleportation of the cognitiveinformation encoding (such as information-modules viewed from a point or heard or seen as words, images or sounds, or other linguistically-meaningful replications). Here the use of neuronal gain factors and other paramters intended to evaluate changes in attention directed to a particular feature is automatically related to the inherent baseline-firing rate of the neuron. The attended feature y does not need to be present in the stimulus used to map the perception.

### Error Resolution

Events are timed based on the relative perceptual constancy under changing conditions (e.g., shape, size, colour, lightness, distance, location, or pitch/loudness in music, co-articulation, tempo, age/sex/dialect/etc of a speaker). Error in the teleportation routine can be evoked by visual patterns that may be too ambiguous for the system to recognize or for the source-receiver to recognize with one unique interpretation per module. Transitions from one percept to its alternatives known as perceptual reversals are spontaneous and stochastic events that cannot be eliminated by intentional effort. This varies even more so in the presence of mental disorders (e.g., bipolar) or in situations where the invention is used to trigger correspondence or evaluation with unborn foetus.

### BRIEF DESCRIPTION OF DRAWINGS

FIG. 1.A and B are schematic process diagrams of the invention

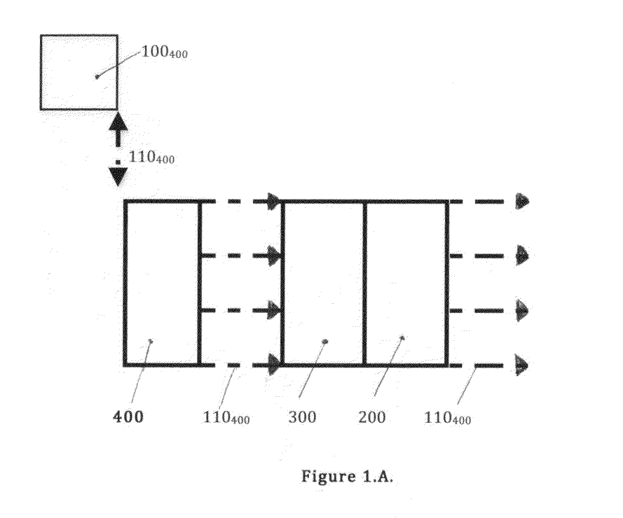

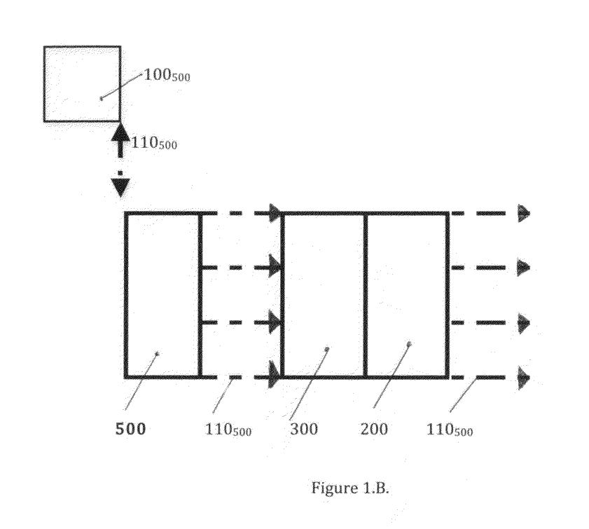

### SUMMARY

The entire assembly can be embedded in a contact lens or electronic speaker ear buds. In part, the invention is based on the realization that the ideal demonstration of priority perceptions takes place at primary sensory organs where the collection of nerves are used by the brain to re-produce or simulate or logify the experience of primary attention to the subject. Losses can be reduced by using methods of converting information encoded on the electromagnetic waves by time-dependent modules and time delays associated with time-bin encoding, such as described in patent application US 2008/0198437 A1 or similar.

In summary, the features of the invention include:

Radio-telephone modulation assembly (100) which provides a method of transceiving information detected by the invention assembly at the source subject source to the receiver-subject source and from the receiver-subject source. Existing radio-frequency waves such as from a mobile phone at the rate of 900 MHz GSM/1800/2100 MHz (in European system) or 824-894 MHz CDMA currently to the appropriate detection rate. Radio-waves carry and transmit digital instructions within the pulsing action. They are available universally through the source subject at fixed pulse rates no different than those used in MRI evaluations for initiation and mobile phone transceiver activation. MRI systems use a base radio frequency rate typically around 45-46 MHz by comparison. Startup will ideally include music patterns/teaching activation to activate the receiver's cortex and corresponding neuronal processing for invention readiness. Neuronal processing of the converted “digital data” from the inner ear electrical signals means that the device inversely delivers information converted through the rest of the assembly into acoustically-enabled information coding that are available for subsequent immediate carriage by the mobile phone device or other display/storage conversions. These same modulated radio-telephone frequency waves permeate and provide the same consistent purpose for optical and foetal sensory information deductions.

Converter assembly (200) hosts the bulk of information conversion between receiver- and subject-sources, based on integrated radio signal configurations and transformations derived with the signals sent according to the presence or absence of incoming electromagnetic pulse arrangements inherent to each perception event. The assembly represents transmission, processing and storage at the nano-second event level. Data coming in is held from sensors that are part of the input subsystems in the Detector Conference System that lies previous or following. The conversion effects transformations that are produced in a series, much like or similar to a Fibonacci arrangement containing differing polarization states of waves having components at different space and time locations. The assembly construction can incorporate arrays of delay-modules and transmitters based on the position of photons in different time bin states before transmission to the receiver-source subject. Its design can accommodate auxiliary signals into the transmitter much like those described in the same patent US 2008/0198437 A1. The lens or ear-plug based conversion assembly contains the electric circuit as well as indicator light-emitting diodes for on/off display. The lenses have no adverse effect on the activity of the wearer but need to be placed in both eyes (where both eyes are functional). Ideally, installing or removing the lens does not affect wearer or re-usability. The flexible organic materials used in contact lenses means that the circuits are to be built using microfabrication techniques reknown for self-assembly capacities in addition to capillary—force action pull the layers of metal and magnetic components (only a few nanometers thick) into position. Computation of the information encoding on the electromagnetic waves can take place using nano-scale and multi-scale computational architectures as described in U.S. Pat. No. 7,535,070 (Eshahian-Wilner, et al) comprised of a plurality of inputs and a plurality of outputs in a device configured to simultaneously transmit data elements from the inputs to the outputs by using spin-waves of different frequencies. Such an architecture incorporates corresponding spin-wave crossbars, reconfigurable mesh, fully-interconnected cluster, a hierarchical multi-scale crossbar and reconfigurable mesh and fully-interconnected cluster arrangements. There is not much separation or distinguishing between the Detector Conference System and the Conversion processes that will follow or precede in the invention (depending on the mode of operation). Such devices are configured to simultaneously transmit a plurality of input data elements to the outputs by using spin-waves of different frequencies.

Detector Conference Systems (300) work directly with the source (400) such as the eye, the ear, the developing brain-nervous system regions of a foetus, or other primary sensory organ) and with the principles of quantum mechanics found in, for example, U.S. Pat. No. 7,184,555 B2. Here the control circuitry is applied in the same manner but using materials with higher magnetic sensitivity to the incoming information encoding (spinors). Distribution of the information follows arrangements belonging to spin-wave architectures found in U.S. Pat. No. 7,535,070 or similar. The invention is scanless and as a result produces a scanless retinal-neuronal perception display system that is ready t produce sub-systems of deliverables for conversion, storage and display. This is derived from manipulation of the Riemannian or Twistor or other quantum mechanical geometry-principles aid in the readiness for uptake of cognitive information encoded on the electromagnetic waves from sensory neurons at the location of the primary sensory organ, as stated above.

1. a method of teleportation of the quantum nature of cognitive information content encoded on the electromagnetic radiation waves present in-, on- or near—as well as in direct association to primary sensory perception in a subject-source to a receiver-source and/or peripheral computation device and/or other receiving device.

2. a method of scanless information conferencing of a plurality of cognitive information content encoded on the electromagnetic radiation waves present in-, on- or near—as well as in direct association to primary sensory perception.

3. a method of decoding a plurality of output relationships between subject-source emotion and perception-activation values

4. a method for incorporating radio-frequency with the assembly-method for real-time communication of visual-/acoustic-sensory cognitive perceptions.

5. The method in claim #1, #2, #3, and #4
a) a scanless mirroring component capable of detecting and transmitting concentrated localizations of information encoded electromagnetic activity within and running into primary sensory organs for perception-based cognitive functioning with radio-telephone frequency applications
b) a scanless mirroring component capable of detecting and transmitting concentrated localizations of information encoded electromagnetic activity within and running into primary sensory organs for perception-based cognitive functioning without radio-telephone frequency applications
c) a method of delineating the tangible relationship between neuron firing and projective geometries ideally suited for cognitive relationships according to complex Hilbert, Minkowski or other quantum mechanical space-time models in order to transfer arrays of perceptual-information encodings from the brain processes

6. As in claim 5, an assembly comprising Detector Conference System and/or Converter Assembly for Source-subject source

7. As in claim 5, an assembly comprising Convertor Assembly and Detector Conference System for Receiver-subject source

8. As in claim 1 through 6, an assembly comprising both Detector Conference System and Converter Assembly for both Source-subject and Receiver subject source

9. As in claim 1 through 8, any arrangement of Detector Conference system

10. As in claim 1 through 8, any arrangement of Converter Assembly
